(19) United States
(12) Patent Application Publication
(10) Pub. No.: US 2020/0003765 A1
(43) Pub. Date: Jan. 2, 2020

(54) METHOD AND APPARATUS FOR DETECTING AN ANALYTE

(71) Applicant: NDM TECHNOLOGIES LTD, Loughborough (GB)

(72) Inventors: Dewan Fazlul Hoque CHOWDHURY, Loughborough (GB); Stephen MOORE, Loughborough (GB)

(21) Appl. No.: 16/341,707

(22) PCT Filed: Aug. 29, 2017
(86) PCT No.: PCT/GB2017/052524

§ 371 Date: Apr. 12, 2019

(30) Foreign Application Priority Data
Oct. 14, 2016 (GB) 1617476.5

(51) Int. Cl. G01N 33/542; G01N 21/64; G01N 33/58

(52) U.S. Cl. CPC G01N 33/542; G01N 21/6428; G01N 21/6486; G01N 2021/772; G01N 2021/6419; G01N 2021/6441; G01N 33/582

(57) ABSTRACT

A sensor provides a measurement of an analyte such as glucose in a sample via a competitive binding FRET assay. A multi-wavelength radiation source comprises two or more distinct wavelengths such that at least two of the wavelengths are within the absorption band of the fluorescent energy donor of the FRET assay and at least one of those wavelengths is also within the absorption band of the energy acceptor of the FRET assay, giving rise to the phenomenon of spectral bleed-through. Because the degree of bleed-through varies with excitation wavelength, the multi-wavelength source allows the sensor to provide multiple measurement channels, which can be used to reduce errors in the analyte measurement.

### BACKGROUND

The present invention relates to a sensor and a method of using the sensor for measuring the concentration of an analyte. An example of an analyte for sensing is glucose.

The use of assays for measuring analyte concentration through a variation in Fluorescence Resonance Energy Transfer (FRET) (also known as Forster resonance energy transfer) is well known. FRET is the non-radiative transfer of energy from a fluorescent energy donor moiety that is in an excited state to an energy acceptor moiety, resulting in an excited state of the acceptor. FRET can only occur when the donor and acceptor moieties are in close proximity and the strength of FRET is proportional to the 6^{th} power of the separation of the donor and acceptor pair, so the effect can be used as a sensitive measure of their separation.

FRET causes a decrease in lifetime and intensity of the donor fluorescence. If the acceptor is fluorescent, FRET may also cause an increase in the emission of the acceptor. Fluorescent emission from a FRET assay is also depolarised. The degree of FRET can be measured via a change in one or more of the aforementioned fluorescence parameters.

In a simple form of FRET assay, one of the fluorescent energy donor moiety and the energy acceptor moiety is labelled with (i.e. bound to) an analyte receptor. The other of the fluorescent energy donor moiety and the energy acceptor moiety is applied to a sample and becomes bound to any of the analyte that is present in the sample. When the sample thus labelled is introduced to the assay, the analyte binds to the receptor, bringing the donor and the acceptor into close proximity so that FRET occurs between them. The degree of FRET provides a measure of the concentration of the analyte in the sample.

Competitive binding FRET assays are also well known. This form of FRET assay further includes a competing ligand, which competes with the analyte to bind to the receptor. If the sample is pre-treated to label the analyte with the donor or acceptor moiety as previously described, then the competing ligand can be unlabelled. On introducing the sample to the assay, the labelled analyte displaces the competing ligand and binds to the labelled receptor, increasing FRET to a degree that serves as a measure of the concentration of analyte in the sample. Alternatively, the competing ligand may be labelled with the donor or acceptor moiety, which gives the benefit that the sample does not need to be pre-treated. In this arrangement, the assay initially contains the labelled ligand bound to the labelled receptor and FRET occurs. When the sample is introduced to the assay, the unlabelled analyte displaces the competing ligand from the receptor and FRET decreases to a degree that serves as a measure of the concentration of analyte in the sample.

U.S. Pat. No. 5,342,789 describes a method utilising a competitive binding assay for the detection of glucose in bodily fluids via FRET. EP0561653 describes a method of determining the extent of FRET via the donor emission lifetime. EP1828773 describes a similar approach using an animal lectin as a glucose receptor.

All of the above methods and systems describe the excitation of the donor with a single wavelength source as depicted in FIG. 1. Such illumination results in a single value of donor emission intensity, acceptor emission intensity, donor emission lifetime, or proportion of depolarisation that varies with the extent of FRET. A ratio measurement of the donor to acceptor emission intensity can provide a more accurate, robust measurement in the presence of large error sources such as those encountered in biological systems, however excitation with a single wavelength still provides one ratio value. Reliance on variation of a single value leaves a sensor prone to error. The complexity of biological systems can introduce many sources of error into a measurement.

Most of the above methods and systems describe the use of a second wavelength source to exclusively excite the acceptor and provide an additional calibration method, however this only provides a baseline correction.

FRET microscopy has used a multiple channel technique for improved imaging by using more than one FRET donor-acceptor pair with distinctly different wavelength operation bands. However, this requires the use of additional fluorescent label pairs.

Spectral bleed-through is a well understood phenomenon in FRET assays. Acceptor bleed-through occurs when the acceptor absorption band overlaps with the absorption band of the donor and the excitation source contributes to absorption in both the donor and acceptor species. Similarly, donor bleed-through can occur when the emission band of the donor extends into the detected band of emission of the acceptor. The systems and methods described above, along with other typical designs by those skilled in the art, attempt to minimise the extent of both forms of spectral bleed-through. Approaches such as ensuring the absorption bands of the donor and acceptor are sufficiently far apart and choosing the excitation wavelength appropriately are commonplace. Algorithms have been developed to correct for spectral bleed-through in the field of FRET microscopy.

In summary, the accuracy of a single FRET assay in a biological system is limited by the amount of information that can be extracted from a single channel approach. Spectral bleed-through is another source of potential error and FRET assays are typically designed to minimise or compensate for the phenomenon.

### SUMMARY OF INVENTION

Herein is described a sensor and method of use of said sensor to provide a multi-channel measurement of an analyte via a FRET assay. The sensor and method are designed to utilise the phenomenon of spectral bleed-through and its variation with excitation wavelength to provide multiple measurement channels and as a consequence a more accurate analyte measurement.

Specifically, the invention provides a sensor as defined in claim 1.

The invention also provides a method for detecting an analyte in a sample as defined in claim 10.

Preferred but non-essential features of the invention are defined in the dependent claims.

Preferably the multi-wavelength source contains n distinct excitation wavelengths, where n is a number greater than 2 but less than 10. In a preferred embodiment (n−1) wavelengths will lie within the absorption bands of both the donor and acceptor. Each of the n distinct excitation wavelengths will have its own characteristic FRET-dependent (and consequently analyte-dependent) emission response related to the ratio of donor absorption to acceptor absorption at each of the said wavelengths. The ratios of each of then channels can be compared with each other giving rise to n(n−1)/2 ratio values that can be used to determine the extent of FRET and consequent analyte concentration.

In a preferred embodiment the acceptor is a fluorescent energy acceptor and the ratio of acceptor to donor emission is calculated for each of the n excitation wavelengths to give further accuracy in measuring the extent of FRET and the corresponding analyte concentration.

For standard FRET approaches described previously, the value of n is at most 2 giving rise to only a single ratio value, and there is no excitation wavelength that lies within a substantial region of both the absorption bands of the donor and acceptor. For the apparatus described herein the value of n can take any integer value from 2 to 10 giving rise to an array of ratio values ranging in size from 1 to 45. This increased volume of measurement data can be processed with an algorithm to identify and eliminate spurious results arising from sources of error within single measurement channels.

### FRET Assay

The FRET assay is preferably a competitive binding FRET assay as previously described.

The fluorescent donor moiety is a fluorescent dye such as those derived from coumarin, rhodamine, xanthene and cyanine dyes or any other fluorescent species capable of binding to either the analyte receptor or ligand. The acceptor moiety may also be a fluorescent dye such as those described above or it may be a non-fluorescent species such as QSY® 21, which is a carboxylic acid, succinimidyl ester available from Thermo Fisher Scientific (www.thermofisher.com).

The donor-acceptor pair are chosen such that the acceptor absorption band predominantly overlaps the donor emission band and partially overlaps the donor absorption band at a range of wavelengths suitable for donor excitation. In a preferred embodiment the spectral features of the donor and acceptor moieties lie predominantly in the Near Infra-Red (NIR) region which experiences less scattering in human tissue than shorter wavelength light.

The analyte receptor is any analyte binding moiety capable of reversibly binding to the analyte. In a preferred embodiment, the analyte is glucose. Suitable glucose receptors include concanavalin A (ConA), animal lectin, boronic acid derivatives, apo-enzymes and glucose binding proteins.

The competing ligand is any moiety capable of reversibly binding to the analyte receptor in competition with the analyte. If the analyte in the sample is labelled with the donor or acceptor moiety, the competing ligand may be unlabelled analyte. Conversely, if the analyte in the sample is unlabelled, the competing ligand may be analyte that is labelled with the donor or acceptor moiety. Suitable glucose competing ligands include dextran, glucose (if the analyte is labelled), labelled glucose (if the analyte is unlabelled) and other carbohydrate-based moieties.

The donor can be bound to the analyte receptor, in which case the acceptor is bound to the competing ligand or to the analyte in the sample. Alternatively the acceptor can be bound to the analyte receptor, in which case the donor is bound to the competing ligand or to the analyte in the sample.

The medium for the assay may be a hydrogel. The assay may include other materials that can contribute to assay stability, quantum yield or other desirable FRET properties. Other materials may include polymers such as nafion, silicone, natural rubber, synthetic rubber, and other polymers known in the state of the art that allow diffusion of molecules through their matrix.

### Multi-Wavelength Source

The multi-wavelength source is used to excite the donor moiety of the FRET assay in the sensor. The multi-wavelength source also excites the acceptor moiety of the FRET assay. The donor and acceptor moieties are chosen so that there is a significant amount of overlap in the donor and acceptor absorption bands giving rise to acceptor bleed-through at the excitation wavelengths provided by the multi-wavelength source. The term multi-wavelength refers to a set of two or more discrete resolvable wavelength sources. In a preferred embodiment the multi-wavelength source consists of an array of laser diodes of differing wavelength, which may be operated in a low duty cycle pulse mode. In an alternative embodiment the multi-wavelength source consists of an array of light emitting diodes of differing wavelengths. In a further alternative embodiment, the multi-wavelength source consists of a broad-band emitter split into an array of discrete wavelength channels. This splitting could be achieved by diffractive optics, optical band-pass filtering, dichroic mirrors or other techniques known in the art for manipulating a white light source into an array of discrete wavelength bands.

In a preferred embodiment, each of the wavelengths of the multi-wavelength source can operate independently such that each wavelength can be used to excite the FRET assay alternately or two or more wavelengths can excite the FRET assay simultaneously.

### Detectors

One or more detectors are used to monitor the emission from the FRET assay. Examples of suitable detectors include photodiodes, avalanche photodiodes, silicon photomultipliers, photomultiplier tubes or other devices capable of detecting fluorescent radiation in a quantitative manner. In a preferred embodiment optical filters are used in conjunction with the detectors to enable separate measurement of the donor and acceptor emission. The filtering may take the form of band-pass or edge-pass filtering through the use of one or more transmission or reflection optical filters.

In a preferred embodiment an additional detector is used to monitor the intensity of the multi-wavelength source. This detector may also utilise optical filtering to selectively monitor the wavelengths of the multi-wavelength source.

The FRET assay may be positioned at the end of an optical fibre, said optical fibre transporting radiation from the multi-wavelength source towards the FRET assay and transporting radiation emitted from the FRET assay towards the one or more detectors.

### Data Processing

Data processing is used to convert the raw data obtained from the detectors into a value for analyte concentration. Suitable circuitry is included to enable said data processing. The data processing can analyse the raw data from the detectors in response to the illumination of the FRET assay with each of the wavelengths of the multi-wavelength source. The data processing consists of a mathematical algorithm or technique that can convert said raw data into an analyte concentration. Examples of a suitable mathematical algorithm or technique include simple comparative analysis, regression techniques (such as principal components analysis and least squares analysis), machine learning, neural network analysis and other techniques suitable for extracting relationships from complex and noise-containing datasets.

Each sensor requires a calibration to train the algorithm to produce accurate analyte concentration for a given raw data set. In a preferred embodiment a universal calibration can be applied to sensors that have the same assay chemistry and multi-wavelength source. In an alternative embodiment, each sensor possesses its own calibration characteristic that can be acquired by the use of the sensor with a known concentration or concentrations of analyte.

### DESCRIPTION OF THE DRAWINGS

FIG. 1: An example of an assay according to the prior art, showing absorption and emission spectrum for both donor and acceptor with a single broad wavelength excitation source and the associated spectral bleed-through.

FIG. 2: An example absorption and emission spectrum for both donor and acceptor for use in the present invention with a multi-wavelength excitation source, each wavelength corresponding to a distinct value of spectral bleed-through.

FIG. 3: Displays an example of the variation in donor/acceptor emission as a function of analyte concentration for a variety of excitation wavelengths

FIG. 4: Displays an example of the variation in emission spectra for the case where acceptor bleed-through is very low at the excitation wavelength

FIG. 5: Displays an example of the variation in emission spectra for the case where acceptor bleed-though is substantial at the excitation wavelength.

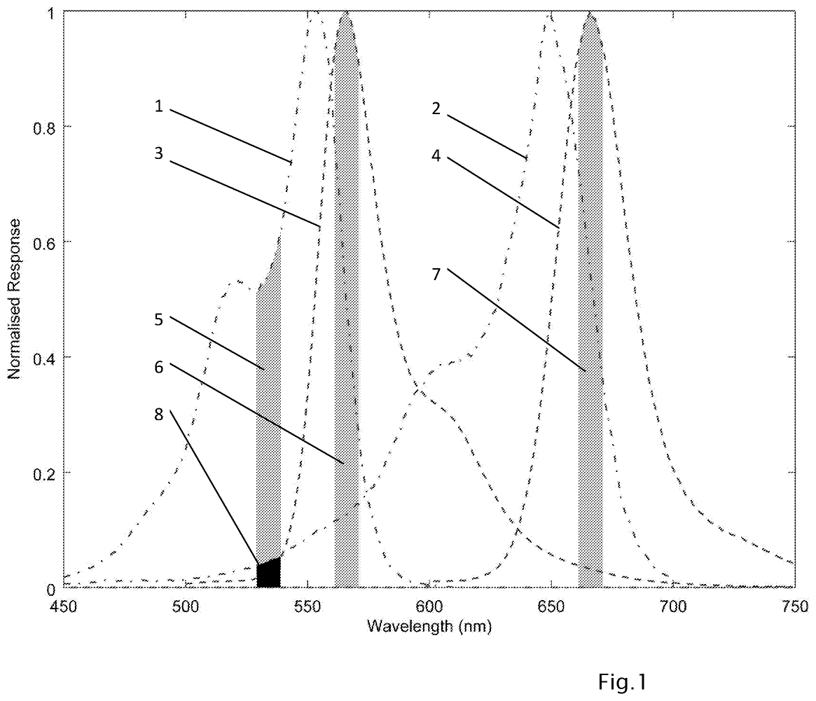

FIG. 1 depicts the use of the spectral response of a FRET assay exhibiting minimal levels of spectral bleed-though as practised by those skilled in the art. The donor absorption 1, acceptor absorption 2, donor emission 3 and acceptor emission 4 are all plotted. The shaded area 5 under the donor absorption curve 1 represents a typical single broad wavelength excitation of the donor such as that provided by a light emitting diode (LED). In this example, the emission of the donor and the acceptor are monitored by filtering the emitted radiation with optical band-pass filters corresponding to wavelengths lying within the shaded regions 6 and 7 for the donor emission and acceptor emission respectively and detecting the filtered emission for each of the donor and acceptor on separate light detectors. In this example there is minimal spectral bleed-through in the form of acceptor bleed-through 8 at the excitation wavelength 5. For a given analyte concentration, only one value of donor emission intensity, acceptor emission intensity, donor/acceptor emission intensity ratio or donor lifetime is obtainable.

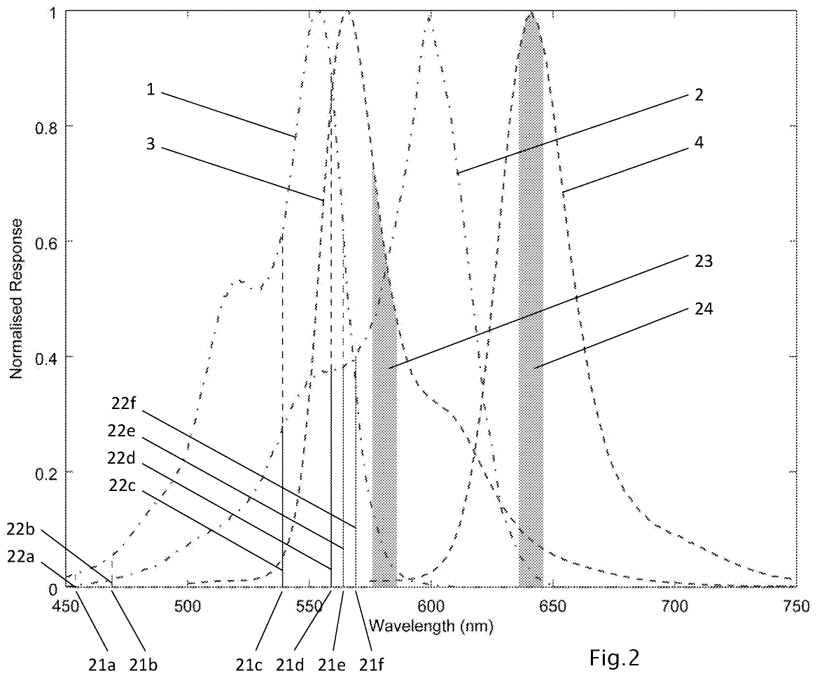

FIG. 2 depicts the use of the spectral response of a similar FRET assay as FIG. 1 but a significant extent of spectral bleed-through and excitation by a multi-wavelength source is indicated, in accordance with the present invention. The multi-wavelength source excites the donor at a series of wavelengths 21a-f. Spectral bleed-through in the form of acceptor bleed-through 22a-f, shown by a solid line below the acceptor absorption curve 2, is present at each of the excitation wavelengths 21a-f, though it is minimal at wavelength 21a, which would be considered outside the absorption band of the acceptor. Each of the excitation wavelengths 21a-f possess a unique ratio of acceptor absorption to donor absorption. As in FIG. 1, the emission of the donor and the acceptor are monitored by filtering the emitted radiation with optical band-pass filters corresponding to wavelengths lying within the shaded regions 23 and 24 for the donor emission and acceptor emission respectively and detecting the filtered emission for each of the donor and acceptor on separate light detectors.

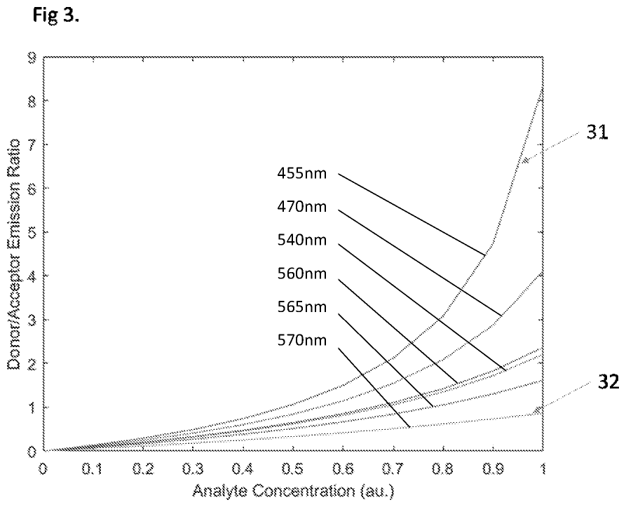

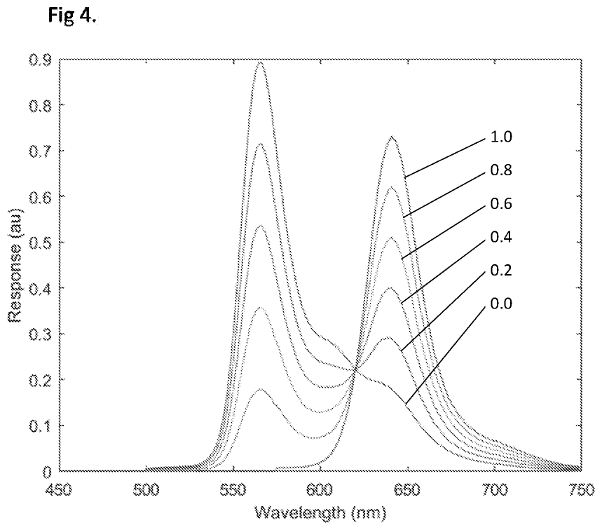

The effect of each of the excitation wavelengths 21a-f on the assay response to analyte concentration is shown in FIG. 3, which plots the donor/acceptor emission ratio for each of the donor excitation wavelengths 21a-f as a function of analyte concentration (stated in arbitrary units). The uppermost curve 31 corresponds to an excitation wavelength 21a equal to 455 nm and an acceptor absorption to donor absorption ratio of 0.12, the lowest value of any of the excitation wavelengths 21a-f and correspondingly to the smallest amount of spectral bleed-through. When the analyte concentration is zero, FRET is dominant and all donor excitation is transferred to the acceptor resulting in zero donor emission. When the analyte concentration is at a saturating level and no FRET is present, the ratio of donor emission to acceptor emission (=8.35) is given by the inverse of the acceptor absorption to donor absorption ratio. The emission spectra for the excitation wavelength 21a are shown in FIG. 4 for varying levels of analyte concentration.

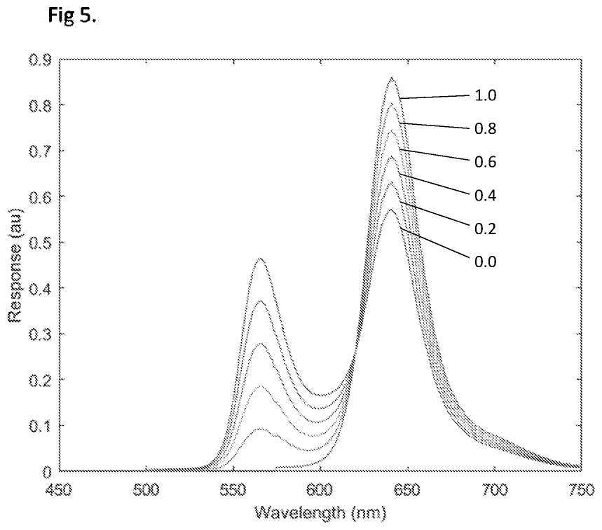

The lowermost curve 32 in FIG. 3 corresponds to an excitation wavelength 21f equal to 570 nm and an acceptor absorption to donor absorption ratio of 1.15, the highest value of any of the excitation wavelengths 21a-f and correspondingly the highest amount of spectral bleed-through. In a similar manner to excitation wavelength 21a, when the analyte concentration is zero, FRET is dominant and all donor excitation is transferred to the acceptor resulting in zero donor emission. When the analyte concentration is at a saturating level and no FRET is present, the ratio of donor emission to acceptor emission is given by the inverse of the acceptor absorption to donor absorption ratio; in the case of excitation wavelength 21f this emission ratio is equal to 0.87. The emission spectra for the excitation wavelength 21f are shown in FIG. 5 for varying levels of analyte concentration.

The excitation wavelengths 21b-e give rise to the intermediate curves of FIG. 3 and have their own corresponding unique sets of emission spectra. The unique variation in response of the assay to each excitation wavelength 21a-f gives rise to a large volume of useable data for determining the analyte concentration. Each wavelength represents an independent channel for determining the analyte concentration. The large volume of independent data and the multiple channels provide a means for accurate determination of analyte concentration even in environments with multiple sources of error such as those present in biological systems. The large volume of data permits the exclusion of erroneous data points arising from sources of measurement error though appropriate data processing as known by those skilled in the art. The sensor contains circuitry for said data processing which may be performed by simple value comparison for each channel, regression analysis, neural network analysis or other mathematical technique capable of extracting an accurate measurement of analyte in the presence of unknown error sources. In a preferred embodiment, a universal calibration would enable the data processing method to work across all sensors with identical assay chemistry and target analyte.

1. A sensor for detecting an analyte comprising:
a) a FRET assay to be placed in contact with a sample containing said analyte, comprising a fluorescent energy donor moiety and an energy acceptor moiety, wherein the absorption band of the said energy acceptor at least partially overlaps both the emission band and absorption band of the fluorescent energy donor;
b) a multi-wavelength radiation source for exciting the said FRET assay, the source comprising two or more distinct resolvable wavelengths such that at least two of the wavelengths are within the absorption band of the fluorescent energy donor and at least one of the wavelengths is within the absorption bands of both the fluorescent energy donor and the energy acceptor;
c) one or more detectors for detecting radiation emitted from the said FRET assay upon excitation by radiation from the said multi-wavelength radiation source; and
d) circuitry containing an algorithm to compare the detected radiation emitted from the said FRET assay when excited by at least one pair of the distinct excitation wavelengths and to calculate the concentration of analyte.

2. The apparatus of claim 1 wherein the energy acceptor is a fluorescent energy acceptor.

3. The apparatus of claim 2 wherein the detectors also detect radiation emitted from the fluorescent energy acceptor.

4. The apparatus of claim 1 wherein the multi-wavelength radiation source comprises multiple laser diodes.

5. The apparatus of claim 1 wherein the analyte is glucose.

6. The apparatus of claim 1 wherein the assay is contained within a hydrogel.

7. The apparatus of claim 1 wherein the FRET assay is positioned at the end of an optical fibre, said optical fibre transporting radiation from the multi-wavelength source towards the FRET assay and radiation emitted from the FRET assay towards the one or more detectors.

8. (canceled)

9. The apparatus of claim 1 wherein the FRET assay is a competitive binding FRET assay.

10. A method of detecting an analyte comprising:
a) placing a sample containing said analyte in contact with a FRET assay, which comprises a fluorescent energy donor moiety and an energy acceptor moiety, wherein the absorption band of the said energy acceptor at least partially overlaps both the emission band and absorption band of the fluorescent energy donor;
b) exciting the said FRET assay with two or more distinct resolvable wavelengths such that at least two of the wavelengths are within the absorption band of the fluorescent energy donor and at least one of the wavelengths is within the absorption bands of both the fluorescent energy donor and the energy acceptor;
c) detecting radiation emitted from the said FRET assay upon excitation by radiation from the said multi-wavelength radiation source; and
d) comparing the detected radiation emitted from the said FRET assay when excited by at least one pair of distinct excitation wavelengths to calculate the concentration of analyte.

11. (canceled)

12. The method of claim 10, further comprising the step of detecting fluorescent radiation emitted from the energy acceptor.

13. The method of claim 10, wherein the analyte is glucose.

14. The method of claim 10, wherein the FRET assay is a competitive binding FRET assay.
